(19) United States
(12) Patent Application Publication
(10) Pub. No.: US 2010/0125261 A1
(43) Pub. Date: May 20, 2010

(54) Disposable Absorbent Articles Comprising Odor Controlling Materials In A Distribution Profile

(76) Inventors: Randall Alan Watson, Loveland, OH (US); Sharon Anne Keegan, Lawrenceburg, IN (US); Edward Joseph Urankar, Mason, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/618,841

(22) Filed: Nov. 16, 2009

Related U.S. Application Data
(60) Provisional application No. 61/116,490, filed on Nov. 20, 2008.

(51) Int. Cl. A61L 15/46

(52) U.S. Cl. 604/359

(57) ABSTRACT

An absorbent article comprising a topsheet, a backsheet, and an absorbent core between the topsheet and backsheet. The absorbent core may comprise an odor control system. The absorbent core has a first quadrant, a second quadrant, a third quadrant, and a fourth quadrant. The odor control system may have a distribution profile greater than 5% in at least two of the four quadrants.

### CROSS-REFERENCE TO RELATED APPLICATIONS

This application claims the benefit of U.S. Provisional Application No. 61/116,490, filed Nov. 20, 2008, which is hereby incorporated by reference in its entirety.

### FIELD OF INVENTION

The present disclosure generally relates to activatable odor control systems and methods for incorporating such systems into disposable absorbent articles.

### BACKGROUND OF THE INVENTION

Absorbent articles, such as disposable diapers, sanitary napkins, pantiliners, incontinence pads, tampons, and the like are typically utilized for absorbing body fluids such as urine, feces, vaginal fluids, and menses. Upon absorbing these fluids, the absorbent articles can be found to contain a number of volatile chemical compounds that include fatty acids (e.g., isovaleric acid), sulfur containing compounds (e.g., mercaptans and sulfides), ammonia, amines (e.g., triethylamine), ketones (e.g., 4-heptanone), alcohols, and aldehydes (decanal) which contribute to the unpleasant odors which can be released from these products during wear or upon disposal. The compounds may be present in the bodily fluids or may develop over time by chemical reaction and/or fluid degradation mechanisms once the fluid has been absorbed into the absorbent article. In addition, once the bodily fluids have been absorbed into the absorbent article, they usually come in contact with microorganisms and/or enzymes that can also generate malodorous by-products as a result of degradation mechanisms such as putrefactive degradation, acid degradation, protein degradation, fat degradation, and the like. These odors can lead to unpleasant experiences for the wearer of the absorbent article and caregiver alike and can make the discreet use and/or disposal of the absorbent articles difficult.

Various odor control materials, agents, techniques, and systems have been disclosed in the art to combat some of the unpleasant odors referred to above, including masking (i.e., covering the odor with a perfume), absorbing the odor already present in the bodily fluids and those generated after degradation, or preventing the formation of the odor. A subset of these various odor control materials, agents, techniques, and systems are activatable. While activatable odor control systems exist in the prior art, disclosure of their effective distribution is lacking. Specifically, due to the unpredictability of where a load will occur along the core, placement of the activatable odor control system becomes critical. Load position, that is, where in the absorbent article bodily fluids will be deposited, is unpredictable due to differences in the way an absorbent article may be applied to the wearer, as well as differences in the anatomy of different wearers.

Another problem related to the effective distribution of activatable odor control systems is having the right amount (enough to combat the odor/odor causing agents) of the system in the place of a low-volume load. Low-volume loads wet only a small portion of the core/diaper. Thus, if the odor control system is not effectively distributed, it will not be successfully activated by the low-volume load. Further, the activatable odor control system should be effectively distributed to deal with multiple loads, or multiple void events. Effective distribution of the odor control system maintains capacity for second, third, or fourth events, whereas a localized odor control system might not be responsive to later events, or might require excessive amounts of an odor control system to respond to later events. This ability of the odor control systems to respond to multiple events over time may be referred to as the stability of the system during use.

For all the reasons above, it would be desirable to provide an activatable odor control system that is effectively distributed in an absorbent article. It would further be desirable to provide enough of the activatable odor control system to adequately and effectively protect against a low-volume load. Still further, it would be desirable to provide for an adequately potent activatable odor control system that is stable through the expected time of use of the product it is placed into.

It has been found that the desired effects can be accomplished by using various activatable odor control systems, including peroxygen bleaching compounds (e.g., sodium percarbonate, sodium perborate, etc.), bleach activator systems (including a combination of sodium percarbonate and sodium nonanoyloxybenzenesulfonate (NOBS)) as disclosed in U.S. Ser. No. 60/989,071, and perfume delivery technologies (e.g., starch encapsulated accords) as disclosed in U.S. Pub. No. 2007/0213412.

### SUMMARY OF THE INVENTION

An absorbent article is disclosed comprising a topsheet, a backsheet, and an absorbent core between the topsheet and backsheet. The absorbent core may comprise an odor control system. The absorbent core has a first quadrant, a second quadrant, a third quadrant, and a fourth quadrant. The odor control system may have a distribution profile greater than 5% in at least two of the four quadrants.

### BRIEF DESCRIPTION OF THE DRAWINGS

FIG. 1 topview of an exemplary absorbent article, with a core that is segmented into four quadrants.

FIG. 2 sideview of an exemplary absorbent article.

### DETAILED DESCRIPTION OF THE INVENTION

As used herein, “distribution profile” means the weight percent of an odor control system/active determined to be present in multiple sections (e.g., quadrants) of an absorbent article.

As used herein, “activatable” refers to an odor control system that requires bodily fluid to trigger the mechanism responsible for combating undesirable odor/odor causing compounds. The activatable odor control system may release or form an odor control active such as a hydrogen peroxide that may remove or inhibit the formation of the odor causing compounds; or the hydrogen peroxide, for instance, may trigger formation of another odor control active such as a peracid if the hydrogen peroxide reacts with a peracid precursor.

As used herein, “non-activatable” refers to an odor control system that, once placed in an absorbent article, requires no further activation. That is, the mechanism of a non-activatable odor control system does not require wetting or other intervention to provide some benefit in terms of reducing malodor.

The disposable absorbent articles according to the present disclosure may comprise an activatable odor control system. The odor control system may comprise one of a peroxygen bleaching compound, a bleach activator system, perfume delivery technologies, or a combination thereof. The bleach activator system may comprise a peroxygen bleaching compound used in combination with a bleach activator described herein after.

The Peroxygen Bleaching Compound

The peroxygen bleaching compounds include those capable of yielding hydrogen peroxide in aqueous liquor. Hydrogen peroxide sources are described in detail in Kirk Othmer's

Encyclopedia of Chemical Technology, 4th Ed (1992, John Wiley & Sons), Vol. 4, pp. 271-300 “Bleaching Agents (Survey),” and include the various forms of sodium perborate and sodium percarbonate, including various coated and modified forms.

The sources of hydrogen peroxide may include any convenient source, including hydrogen peroxide itself. For example, perborate, e.g., sodium perborate (any hydrate including the mono- or tetra-hydrate) or equivalent percarbonate salts, sodium pyrophosphate peroxyhydrate, urea peroxyhydrate, or sodium peroxide may be used. Also useful are sources of available oxygen such as persulfate bleach (e.g., OXONE, manufactured by DuPont). Other useful sources of hydrogen peroxide include stable complexes of polyvinylpyrrolidone with hydrogen peroxide (as disclosed in U.S. Patent App. No. 2006/0292091 and available from International Specialty Products, N.J. under the tradename Peroxydone) and stable crystalline complexes of carbohydrate and hydrogen peroxide (as disclosed in U.S. Pat. No. 6,887,496). Mixtures of any hydrogen peroxide sources can also be used.

A percarbonate bleach may comprise dry particles having an average particle size in the range from about 500 micrometers to about 1,000 micrometers, not more than about 10% by weight of the particles being smaller than about 200 micrometers and not more than about 10% by weight of the particles being larger than about 1,250 micrometers. Sodium Percarbonate, available from OCI Chemical Corp, Decatur, Ala. under the tradename Provox C or Kemira Kemi AB, Sweden under the tradename ECOX-C, can be in uncoated or coated form.

Peroxygen bleaching compounds (e.g., sodium percarbonate) may be coated. Suitable coatings include, but are not limited to, silicate, borate, sulfate, or water-soluble surfactants, as disclosed, for example, in U.S. Pat. Nos. 5,556,834; 6,521,583; and 7,588,697, and WO 97/19890A1. There are advantages to using a coated peroxygen bleaching compound, including stabilizing the peroxygen bleaching compound against decomposition in humid environments or in proximity to other compounds that may accelerate their decomposition (e.g., organic compounds, including perfume raw materials). Such benefits may be provided by a variety of inorganic materials that create at least a partial shell around the peroxygen bleaching compound. The coating material may be soluble in water or bodily fluids to expose the peroxygen bleaching compound after the absorbent article is soiled.

The Bleach Activator

The peroxygen bleach compound may be formulated with a bleach activator. The bleach activator may be considered a “peracid precursor.” The bleach activator may be present within the absorbent article at levels from about 0.001 g, from about 0.005 g, from about 0.01 g to about 0.05 g, to about 0.2 g, to about 1.0 g per absorbent article. The bleach activator may include any compound, which, when used in conjunction with a hydrogen peroxide source, results in the in-situ production of a peracid corresponding to the bleach activator. Examples of bleach activators are disclosed in U.S. Pat. Nos. 5,576,282; 4,915,854; and 4,412,934. U.S. Pat. No. 4,634,551 also discloses peroxygen bleaching compounds and bleach activators.

Bleach activators may include tetraacetyl ethylene diamine (TAED), benzoylcaprolactam (BzCL), 4-nitrobenzoylcaprolactam, 3-chlorobenzoylcaprolactam, benzoyloxybenzenesulphonate (BOBS), nonanoyloxybenzenesulphonate (NOBS), phenyl benzoate (PhBz), decanoyloxybenzenesulphonate (C₁₀-OBS), benzoylvalerolactam (BZVL), octanoyloxybenzenesulphonate (C₈-OBS), perhydrolyzable esters and mixtures thereof, and benzoylcaprolactam and benzoylvalerolactam. Bleach activators that have an OBS or VL leaving group may be used. Hydrophobic bleach activators may be used. Bleach activators are also described in U.S. Pat. Nos. 5,698,504; 5,695,679; 5,686,401; 5,686,014; 5,405,412; 5,405,413; 5,130,045; 4,412,934; and U.S. Ser. Nos. 08/709,072; 08/064,564; and U.S. Ser. No. 60/989,071.

In selecting a bleach activator, the surface activity of the bleach activator should be considered in light of the specific construction of the absorbent article. Higher surface activity may reduce the surface tension of bodily liquids that contact the bleach activator. The reduced surface tension may increase the rewet tendencies of some absorbent articles (that is, the tendency to release previously absorbed liquid), or negatively affect the absorptive capacity of the absorbent article, possibly contributing to leakage events. It should be understood that a “high” surface activity will vary depending on the materials and structure used in the absorbent article, particularly in the absorbent core.

The mole ratio of hydrogen peroxide (neat or as delivered from the peroxygen source) to bleach activator in the present invention may range from at about 100:1 to 1:1; from about 80:1 to 5:1, and from about 70:1 to about 20:1.

Bleach Activator System as a Co-Particle

The peroxygen bleaching compounds and bleach activator materials can be incorporated into the absorbent articles by any means or in any form that allows in-situ generation of the peracid. This could include the addition of the two materials added separately or as a premixed solid to the absorbent article. For instance, the peroxygen bleaching compound and bleach activator may be delivered to the absorbent article as a co-particle composition through the use of a dispersant aid or binder material as disclosed herein or as disclosed in WO 2007/127641.

Bleach Activator System as a Multiple Particle Mixture

The peroxygen bleaching compound and bleach activator may also be delivered to the absorbent article as a mixture of particles. For instance, the bleach activator may be in the form of an extrudate as disclosed in U.S. Pat. Nos. 4,486,327 and 6,617,300 and then mixed with a peroxygen bleaching compound to provide a multiple particle bleach activator system. Additionally, in some embodiments, the bleach activator may be coated onto a core particle through the use of suitable binders and coating materials as disclosed in WO 2005/080542, U.S. Patent Application Publication No. 2006/0252667, and EP 1 881 059 A1, and then mixed with an appropriate amount of peroxygen bleaching compound to provide a multiple particle bleach activator system.

Suitable core materials used to make the bleach activator particle include, but are not limited to, ingredients such as sodium sulfate, sodium carbonate and sodium phosphate, as well as composite detergent ingredient compositions made by processes such as spray-drying, agglomeration, compaction, and/or extrusion processes. Examples of such composite compositions include particles/granules comprising detergent builder, surfactant and, optionally, polymer ingredients. Suitable core materials may have a particle size that is comparable to the peroxygen bleach compound to facilitate proper mixing between the peroxygen bleach compound and the bleach activator particle and may have a particle size that will range from about 200-1300 μm and may have an average particle size from about 500-1000 μm. While suitable cores, such as detergent particles/granules, are typically made as an intermediate within a detergent production facility, suitable cores and core raw materials can be obtained from FMC Corporation of Philadelphia, Pa., U.S.A.; Jost Chemicals of St. Louis, Mo., U.S.A.; General Chemical Corporation of Parsippany, N.J., U.S.A; and Mallinckrodt Baker of Phillipsburg, N.J., USA. Additionally, superabsorbent polymers may also be used as suitable core components and can be obtained from BASF of Ludwigshafen, Germany; Nippon Shokubai of Osaka, Japan; and Evonik Degussa of Düsseldorf, Germany. Further, suitable core materials can be chosen from polymeric particles, inorganic salts, clays, mica, starches, sugars, zeolites, silicon dioxide and inorganic coordination complexes.

Suitable binder materials used to make the bleach activator particle include materials selected from the group consisting of polymers, surfactants, solvents, and mixtures thereof. Examples of polymers include sodium polyacrylate, acrylic-maleic co-polymers, polyethylene glycol, polyvinyl acetate, polyvinyl pyrrolidone, cellulose ethers, and hydroxypropyl cellulose. Examples of surfactants include anionic, cationic, zwitterionic and nonionic surfactants. Examples of solvents include water, alcohols, linear alcohols, branched alcohols, and fatty alcohols. Suitable binders can be obtained from BASF of Ludwigshafen, Germany; Dow Chemical Company of Midland, Mich., U.S.A.; Hercules Incorporated of Wilmington, Del., U.S.A.; Shell Chemical LP of Houston, Tex., U.S.A.; Procter & Gamble Chemicals of Cincinnati, Ohio, U.S.A.; and Rohm and Hass Company of Philadelphia, Pa., U.S.A.

Suitable solid coating aids used to make the bleach activator particle include materials selected from the group consisting of acetates, sulfates, carbonates, borates, phosphates, and mixtures thereof. Examples of acetates include magnesium acetate, Mg(CH₃COO)₂; and sodium acetate, NaCH₃COO. Examples of sulfates include magnesium sulfate, MgSO₄; and sodium sulfate, Na2SO₄. Examples of carbonates include sodium carbonate, Na₂CO₃; potassium carbonate, K₂CO₃. Examples of borates include sodium borate, Na₂B4O₇. Examples of phosphates include sodium phosphate dibasic, Na₂HPO₄; and sodium tripolyphosphate, Na₅P₃OIO. Such coating aids may be introduced to the coating process as substantially anhydrous salts. While not being bound by theory, it is believed that their conversion to stable hydrate phases provides a mechanism for the removal of binder moisture and enables processing without the requirement of a drying step. Suitable solid coating aids can be obtained from PQ Corporation of Valley Forge, Pa., U.S.A.; FMC Corporation of Philadelphia, Pa., U.S.A.; and Mallinckrodt Baker, Inc. of Phillipsburg, N.J., U.S.A.

In addition, the bleach activator particle may optionally comprise dyes and pigments for the purpose of conveying a signal to the caregiver. The signal may communicate the presence of the bleach activator particle. Non-limiting examples of dyes and pigments include organic and inorganic pigments, aqueous and other solvent-soluble dyes. Such dyes and pigments can be obtained from Ciba Specialty Chemicals Corporation of Newport, Del., U.S.A.; Clariant Corporation of Charlotte, N.C., U.S.A.; and Milliken Chemical Company of Spartanburg, S.C., U.S.A. Suitable equipment for performing the particle making processes disclosed herein includes paddle mixers, ploughshare mixers, ribbon blenders, vertical axis granulators, and drum mixers, both in batch and, where available, in continuous process configurations. Such equipment can be obtained from Lodige GmbH (Paderborn, Germany), Littleford Day, Inc. (Florence, Ky., U.S.A.), Forberg AS (Larvik, Norway), Glatt Ingenieurtechnik GmbH (Weimar, Germany). Further, the bleach activator particle may also optionally be dried to remove any moisture prior to being mixed with the peroxygen bleach compound of the multiple particle bleach activator system.

In some embodiments, said bleach activator particle comprises, based on total particle weight, no more than about 50 weight percent of any bleach activator, no more than about 20 weight percent of any bleach activator active, no more than about 10 weight percent of any bleach activator active, or no more than about 5 weight percent of any bleach activator active.

A multiple particle bleach activator system may be formed by mixing the peroxygen bleach compound with the bleach activator particle. Suitable equipment for performing the mixing process includes, but is not limited to paddle mixers, such as a Forberg mixer and rotating drum mixers.

Optional Agents

Dispersant aids or binders may be used. These materials may be used to aid in distributing the bleach activator systems through out the entire core of the absorbent article while also aiding in keeping the peroxygen bleaching compound and bleach activator closely associated with one another. Dispersant aids may be low melting temperature solids to enable mixing with the bleach activator materials and may be hydrophilic to permit sufficient wetting and activation of the peroxygen bleach compounds. Dispersant aids may include glucose, sorbitol, maltose, glucamine, sucrose, polyvinyl alcohol, starch, alkyl polyglycoside, sorbitan fatty ester, polyhydroxy fatty acid amides containing from about 1 to about 18 carbon atoms in their fatty acid moieties, and mixtures thereof. Dispersant aids may also include a polyethylene glycol polymer available from The Dow Chemical Company, Midland Mich. under the tradename Carbowax.

Additional non-activatable odor control systems/materials (e.g., adsorbent materials such as activated carbon; non-encapsulated perfumes) may be used in conjunction with the activatable odor control system of the present disclosure. These materials may be classified according to the type of odor the agent is intended to combat. Odors may be chemically classified as being acidic, basic, or neutral. Alternatively, the odor control agents may be categorized with respect to the mechanism by which the malodor detection is reduced or prevented. For example, odor control agents may chemically react with malodorous compounds or with compounds that produce malodorous degradation products thereby generating compounds lacking odor or having an odor acceptable to consumers. Carbonates (e.g., sodium carbonate), bicarbonates (e.g., sodium bicarbonate), phosphates (e.g., sodium phosphate), sulphates (e.g., zinc and copper sulphates), carboxylic acids such as citric acid, lauric acid, boric acid, adipic acid and maleic acid, zinc salts of carboxylic acids such as zinc ricinoleate, transition metals, activated carbons, clays, zeolites, silicas, superabsorbent polymers, and starches may be used, for example. Such odor control agents and systems are disclosed in EP-A-348 978; EP-A-510 619; WO 91112029; WO 91111977; WO 91112030; WO 81101643; and WO 96106589.

Absorbent Article

Disposable absorbent article 130 may comprise generally chassis 30 and absorbent core 20. Chassis 30 includes topsheet 120 having garment facing surface 160 and body facing surface 150, and backsheet 140 having garment facing surface 180 and body facing surface 170. Absorbent core 20 may be disposed between body facing surface 170 of backsheet 140 and garment facing surface 160 of topsheet 120. Absorbent core 20 has front edge 40 and rear edge 190, with length 10 between front edge 40 and rear edge 190. Front edge 40 is the edge of absorbent core 20 that would be closest to a user's front or ventral side and rear edge 190 is the edge of absorbent core 20 that would be closest to a user's back or dorsal side, when absorbent article 130 is fitted to a user. Length 10 of absorbent core 20 can be divided into quadrants 50, 60, 70, and 80 along lines 90, 100, and 110. As shown, quadrant 50 is nearest front edge 40 and might be described as the front quadrant, where quadrant 60 would be the front-middle quadrant, quadrant 70 would be the back-middle quadrant, and quadrant 80 would be the back quadrant.

In certain embodiments, the absorbent articles may take the form of a diaper, a pant product, an adult incontinence product, or a feminine hygiene product, e.g., a sanitary napkin or panty liner. Absorbent article 130 may have different shapes or sizes within and between different forms. Given these various product forms, additional components may also exist within the disposable absorbent article. Such components may be selected from the group consisting of an outer cover, side panels, a cuff, an elastic feature, a wing, a fastening system, and combinations thereof. Other additional components are possible, as known in the art.

Absorbent Core

The articles of the present disclosure may additionally comprise one or more absorbent cores 20. Absorbent core 20 is at least partially disposed between topsheet 120 and backsheet 140 and may take on any size or shape that is compatible with the disposable absorbent article. Exemplary absorbent structures for use as absorbent core 130 that have achieved wide acceptance and commercial success are described in U.S. Pat. Nos. 4,610,678; 4,673,402; 4,888,231; and 4,834,735; and U.S. Pub. Nos. 2005-0273071, 2005-0171499, 2007-0191806, 2004-0162538, and 2005-0095942. The absorbent core may further comprise a dual core system containing an acquisition/distribution core of chemically stiffened fibers positioned over an absorbent storage core as detailed in U.S. Pat. Nos. 5,234,423 and 5,147,345.

As discussed herein “absorbent gelling materials” and “superabsorbent polymers” are those materials that, upon contact with aqueous fluids, such as bodily fluids, imbibes such fluids and form hydrogels. These absorbent gelling materials are typically capable of absorbing large quantities of aqueous bodily fluids, and further capable of retaining such absorbed fluids under moderate pressures. These absorbent gelling materials are typically in the form of discrete, nonfibrous particles. Other forms, such as fibers, foams, sheets, strips, or other macrostructures, are also suitable for use herein. Suitable absorbent gelling materials in the form of open cell foams are described in U.S. Pat. Nos. 3,563,243; 4,554,297; 4,740,520; and 5,260,345.

In certain embodiments of the present disclosure, the absorbent article may also include a sublayer disposed between topsheet 120 and backsheet 140. The sublayer may have a body facing surface and a garment facing surface and may be any material or structure capable of accepting, storing or immobilizing bodily exudates. Thus, the sublayer may include a single material or a number of materials operatively associated with each other. Further, the sublayer may be integral with another element of the absorbent article or may be one or more separate elements joined directly or indirectly with one or more elements of the article. Further, the sublayer may include a structure that is separate from absorbent core 20 or may include or be part of at least a portion of absorbent core 20.

Additionally, suitable absorbent cores 20 may contain reduced amounts of cellulosic airfelt material. For instance, such cores may comprise less than about 40%, 30%, 20%, 10%, 5%, or even 1%. Such a core comprises primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; 6,790,798; and U.S. Patent Application Publications 2004/0158212A1; 2004/0097895A1; 2004/0158214A1; and 2004/0158213.

In further embodiments, the articles according to the present disclosure may further comprise a wetness sensation member. This member may be disposed in various locations within the article. For instance, the wetness sensation member may be disposed on the topsheet. The member may comprise a permeable layer and an impermeable layer, wherein urine passes through the permeable layer and not through the impermeable layer such that a wearer is made of aware of the fact that urination has occurred as a result of the “wet” feeling. Suitable members are detailed in U.S. Pat. No. 6,627,786.

Bleach activator systems may be incorporated into the absorbent articles described, for example, in U.S. Pub. Nos. 2005/0273071, 2005/0171499, 2007/0191806, 2004/0162538, and 2005/0095942. For example, bleach activator systems may be placed in or on the different absorbent article components described above, including an absorbent core, an acquisition system, barrier leg cuffs, a backsheet, and/or a topsheet. Further, they may be incorporated into lotions applied to the topsheet. These lotions may be hydrophilic or hydrophobic. For instance, bleach activator systems may be suspended in the lotion.

The activatable odor control systems of the present disclosure may be distributed into the absorbent core in a number of distribution profiles, including the core profiles outlined in Table 1.

TABLE 1

| | | Front- | Back- | | |
|---|---|---|---|---|---|
| | Front | Middle | Middle | Back | |
| | Quadrant | Quadrant | Quadrant | Quadrant | Total |
| Core Profile 1 | 25% | 25% | 25% | 25% | 100% |
| Core Profile 2 | 31% | 34% | 25% | 10% | 100% |
| Core Profile 3 | 25-35% | 30-40% | 20-30% | 5-15% | 100% |
| Core Profile 4 | 25-35% | 30-40% | 20-30% | 0-5% | 100% |
| Core Profile 5 | 30-55% | 30-55% | 0-20% | 0-10% | 100% |
| Core Profile 6 | 0-10% | 0-20% | 30-55% | 30-55% | 100% |
| Core Profile 7 | 0-50% | 0-50% | 0-50% | 0-50% | 100% |
| Core Profile 8 | 25-35% | 25-35% | 25-35% | 0-10% | 100% |

Particle metering and pulsing devices suitable for the delivery of superabsorbent polymers used in the production of absorbent articles are known in the art. For instance, suitable methods and apparatuses for metering and pulsing of superabsorbent particles are disclosed in U.S. Pub. No. 2003-0225382. These same methods and apparatuses can be used to meter activatable odor control systems of the present disclosure. The disclosed odor control systems can be metered and pulsed alone, or in combination with a stream of superabsorbent polymer particles. Particularly, a particle metering apparatus that may be used with odor control systems of the present disclosure includes a compact loss-in-weight feeder, type K-ML-SFS-BSP-100 available from K-tron Process Group, Gelnhausen, Germany. When the odor control system particles are metered into the absorbent article in combination with superabsorbent polymer particles, they may be metered into the stream of superabsorbent polymer particles immediately prior to entry into the pulsing device.

### Test Methods

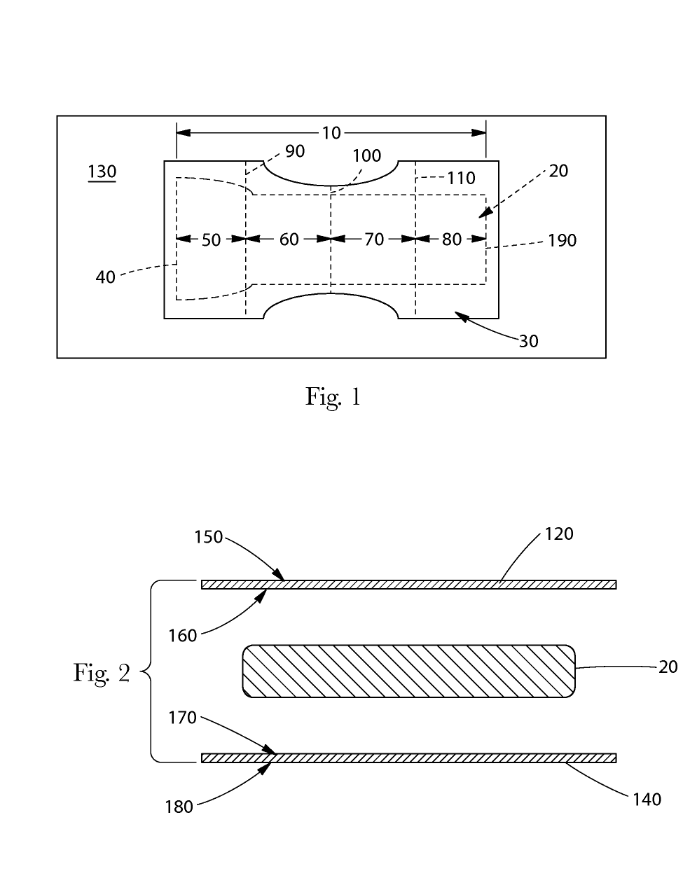

Method of Dividing the Absorbent Article into Quartiles (See FIG. 1)

Stretch the absorbent article to 100% extension on a stretch board, topsheet side up. Measure the overall length (L) of the absorbent core to the nearest 1/10^{th} inch. For the purpose of this method, the absorbent core edge is located at the edge of the absorbent material that makes up the absorbent core (e.g., superabsorbent particles and/or cellulosic fibers), and does not include any extra length contibuted by a core wrap or other sublayer associated with the absorbent material. Calculate:

A (in.)=0.25×L (in.)

B (in.)=0.50×L (in.)

C (in)=0.75×L (in.)

Measure from the front edge of the absorbent core, mark off lengths A, B, and C, and draw a line across the core at each point, parallel to the front edge. Cut the core into four sections by carefully cutting along the lines drawn at A, B, and C. Weigh each section and record the weight as Quandrant 1—F25%, Quadrant 2—FM25%, Quadrant 3—BM25%, and Quadrant 4—B25%, respectively. Discard any material that falls out of the diaper during cutting as it is not possible to determine which portion of the diaper this loose material came from.

### Absorbent Article Extraction—Nonanoyloxybenzenesulfonate Bleach Activator Analysis

Stock extraction solvent is first prepared by mixing 1200 g of 50% aqueous ethanol and 300 g glacial acetic acid in a 2 liter glass bottle to provide a 2:1:2 by weight mix of Denatured Ethanol:Glacial Acetic Acid:Dionized Water solution.

A representative absorbent article is prepared for extraction by carefully removing the cuff, if present, and top edges of the absorbent article, if present, beyond the core and cutting the core into small pieces (approximately 20-1.5 square inch pieces) over a tray or release paper to catch any materials lost from core. The absorbent article material is placed in a 16 oz high density polyethylene wide mouth jar (available from VWR International; Catalog #15900-106) and treated with 200 mL of stock extraction solvent. The lid (equipped with Plastisol liner; available from VWR International; Catalog #16198-905) is placed tightly on the jar to ensure no solution is lost and the jar is placed on a US Stoneware roller type jar mill (available from VWR international; Catalog #48900-000) on a setting of 10 for 30 minutes. The jars are placed inside a disposable nitrile glove (such as those available from VWR International; Catalog #40101-348) to help maintain proper contact with the rollers of the mill and ensure proper mixing. The extraction solvent is collected by squeezing the absorbent article material within the jar and pouring the resulting liquid into a collection container. The extraction solvent is placed in a refrigerator until analysis and should be conducted within 3 days.

### High Performance Liquid Chromotagraphy (HPLC) Test for Nonanoyloxybenzenesulfonate Bleach Activator

A suitable test method for the quantification of Nonanoyloxybenzenesulfonate (NOBS) either from particles coated with NOBS or an absorbent article containing such particles, is by HPLC with UV detection. Analysis is conducted on an Waters 2695 liquid chromatograph solvent delivery system or equivalent, equipped with an in-line degasser, autosampler, column heater (set at 35° C.) and a photodiode array detector and supported by Empower software for instrument control, data collection and processing. Chromatography is performed on a Dionex Acclaim® Polar Advantage reverse phase column (C16 3 micron 150×4.6 mm Part #061318) using a binary gradient, at 1 mL/minute flow rate, with UV detection at 220 nm. Extraction of an absorbent article is described above. The extract is filtered through a 0.45 micron PTFE Acrodisc CR filter before 5 μL is injected for analysis.

### Reagents and Solutions:

Extraction Solvent: Denatured (95%) Ethanol:Glacial Acetic Acid:Water (2:1:2 by weight)
Bleach activator standard: Nonanoyloxybenzenesulfonate (NOBS) powder of known activity
HPLC Eluent A—Aqueous 0.01 M Ammonium Dihydrogen Orthophosphate (HPLC grade)
HPLC Eluent B—70:30 HPLC Grade Acetonitrile:Water (HPLC grade)

### HPLC Gradient Elution Profile

| Time | Eluent A | Eluent B | Flow |
|---|---|---|---|
| (min) | (%) | (%) | (mL/min) |
| 0 | 70 | 30 | 1.00 |
| 10 | 5 | 95 | 1.00 |
| 12.5 | 5 | 95 | 1.00 |
| 13 | 70 | 30 | 1.00 |
| 18 | 70 | 30 | 1.00 |

### Preparation of Calibration Standards:

All standards are prepared using Class A volumetric glassware. First, approximately 100 mg of the NOBS primary standard is accurately weighed and transferred to a 100 mL volumetric flask, brought to volume with the extraction solvent and mixed thoroughly. Next, calibration standards of 10.0, 20.0, 40.0 and 80.0 mg/L are prepared by pipeting 1.00, 2.00, 4.00 and 8.00 mL of the stock solution, each into a separate 100 mL volumetric flask, brought to volume with the extraction solvent, and mixed thoroughly. If needed, additional calibration standards can be prepared to assure that the concentration of the NOBS falls within the span of the calibration curve.

### HPLC Analyses:

5 μL of each calibration standard and the extracted sample is injected. The integrated peak areas for the calibration standards are used to prepare a calibration curve of Response (peak area) verses Concentration from which the concentration of NOBS can be calculated. Report results to ±0.1 mg/mL. This value can be used to calculate the total NOBS extracted from an absorbent article.

mg/absorbent article=measured NOBS concentration (mg/L)*extraction volume (L)/diaper
Results are reported to ±0.1 mg/diaper.

### Absorbent Article Extraction—Tetraacetylethylenediamine Bleach Activator Analysis

Stock extraction solvent is first prepared by mixing 1200 g of 50% aqueous ethanol and 600 g glacial acetic acid in a 2 liter glass bottle to provide a 1:1:1 by weight mix of Denatured Ethanol:Glacial Acetic Acid:Dionized Water solution.

A representative absorbent article is prepared for extraction by carefully removing the cuff, if present, and top edges of the absorbent article, if present, beyond the core and cutting the core into small pieces (approximately 20-1.5 square inch pieces) over a tray or release paper to catch any materials lost from core. The absorbent article material is placed in a 16 oz clear glass wide mouth jar (available from VWR International; Catalog #89043-274) and treated with 200 mL of stock extraction solvent. The lid (equipped with Plastisol liner; available from VWR International; Catalog #16198-905) is placed tightly on the jar to ensure no solution is lost and the jar is placed on a US Stoneware roller type jar mill (available from VWR international; Catalog #48900-000) on a setting of 10 for 30 minutes. The jars are placed inside a disposable nitrile glove (such as those available from VWR International; Catalog #40101-348) to help maintain proper contact with the rollers of the mill and ensure proper mixing. The extraction solvent is collected by squeezing the absorbent article material within the jar and pouring the resulting liquid into a collection container. The extraction solvent is placed in a refrigerator until analysis and should be conducted within 3 days.

### High Performance Liquid Chromotagraphy (HPLC) Test for Tetraacetylethylenediamine Bleach Activator

A suitable test method for the quantification of Tetraacetylethylenediamine (TAED) either from particles coated with TAED or an absorbent article containing such particles, is by HPLC with UV detection. Analysis is conducted on an Waters 2695 liquid chromatograph solvent delivery system or equivalent, equipped with an in-line degasser, autosampler, column heater (set at 35° C.) and a photodiode array detector and supported by Empower software for instrument control, data collection and processing. Chromatography is performed on a Dionex Acclaim® Polar Advantage reverse phase column (C16 3 micron 150×4.6 mm Part #061318) using a binary gradient, at 1 mL/minute flow rate, with UV detection at 220 nm. Extraction of an absorbent article is described above. The extract is filtered through a 0.45 micron PTFE Acrodisc CR filter before 5 μL is injected for analysis.

### Reagents and Solutions:

Extraction Solvent: 50% Aqueous Ethanol:Glacial Acetic Acid (2:1 by weight)
Bleach activator standard: Tetraacetylethylenediamine (TAED) powder of known activity
HPLC Eluent A—Aqueous 0.01 M Ammonium Dihydrogen Orthophosphate (HPLC grade)
HPLC Eluent B—70:30 HPLC Grade Acetonitrile:Water (HPLC grade)

### HPLC Gradient Elution Profile

| Time | Eluent A | Eluent B | Flow | |
|---|---|---|---|---|
| (min) | (%) | (%) | (mL/min) | Curve |
| 0 | 95 | 5 | 1.00 | linear |
| 8 | 5 | 95 | 1.00 | linear |
| 10.5 | 5 | 95 | 1.00 | linear |
| 11 | 95 | 5 | 1.00 | linear |
| 15 | 95 | 5 | 1.00 | linear |
| 18 | 95 | 5 | 1.00 | linear |
| 19 | 95 | 5 | 0.00 | |

### Preparation of Calibration Standards:

All standards are prepared using Class A volumetric glassware. First, approximately 100 mg of the TAED primary standard is accurately weighed and transferred to a 100 mL volumetric flask, brought to volume with the extraction solvent and mixed thoroughly to dissolve. Next, calibration standards of 10.0, 20.0, 40.0 and 80.0 mg/L are prepared by pipeting 1.00, 2.00, 4.00 and 8.00 mL of the stock solution, each into a separate 100 mL volumetric flask, brought to volume with the extraction solvent, and mixed thoroughly. If needed, additional calibration standards can be prepared to assure that the concentration of the TAED falls within the span of the calibration curve.

### HPLC Analyses:

5 μL of each calibration standard and the extracted sample is injected. The integrated peak areas for the calibration standards are used to prepare a calibration curve of Response (peak area) verses Concentration from which the concentration of TAED can be calculated. Report results to ±0.1 mg/mL. This value can be used to calculate the total TAED extracted from an absorbent article.

mg/absorbent article=measured TAED concentration (mg/L)*extraction volume (L)/diaper
Results are reported to ±0.1 mg/diaper.

### Reflectoquant Peroxide Test—Absorbent Article Percarbonate Loading

### Equipment:

Reflectoquant Meter; RQflex 10, available from EMD Chemicals
Peroxide Test Strips; stock number 16974; measuring 0.2-20.0 mg/mL concentration range

### Method:

For the peroxide measurement, the Reflectoquant is used for the analysis with test strips as directed by instructions. A representative absorbent article containing percarbonate is prepared for analysis by carefully removing the cuff, if present, and top edges of the absorbent article beyond the core, if present, and cutting the remaining absorbent article into small pieces (approximately 20-1.5 square inch pieces) over a tray or release paper to catch any materials lost from core. The cut up absorbent article and material is transferred to a 600 ml beaker until analysis. A 2.5 gallon plastic bucket is filled with 4 liters of deionized water at room temperature and a bench top stir motor with an 18′ stainless steel shaft and 1½″ propeller blades is set-up next to bucket to provide agitation to water solution in the bucket. The stirring is begun at a moderate speed and the absorbent article pieces are transferred all together into the 4 liters of deionized water. Upon addition of the diaper pieces, the timer is started and after 25 minutes the agitation speed is turned to high. Reflectoquant readings are taken after 30 minutes by dipping the peroxide test strip directly into the stirred solution containing peroxide, holding it there for 2 seconds prior to removing from the solution and shaking the strip to get rid of excess solution. At the same time the test strip is placed in the solution, the 15 second timer on the Reflectoquant Meter is activated. After removing the excess solution from the test strip, it is inserted into the machine and at the end of the 15 second timer, the reflectoquant analyzes the test strip and displays the concentration of peroxide (ppm) within the solution. At the 30 minute time period, 3 consecutive test strips are analyzed and the readings recorded and averaged.

To determine the loading of sodium percarbonatate within a representative absorbent article, a response curve is first constructed by loading known amounts of sodium percarbonate near the targeted loading level (i.e., 50, 100, 200, 400 mg per absorbent article) into representative absorbent articles (known to contain no percarbonate prior to the loading), and determining the concentration of peroxide using the reflectoquant analysis described above. The response curve is constructed such that the weight of percarbonate in the absorbent article is graphed against the concentration of peroxide determined using the reflectoquant analysis and the data subjected to line-fit analysis. The resulting line and equation is then used to determine the unknown loading of sodium percarbonate in line made absorbent articles.

### Calculate the Distribution Profile—Peroxygen Bleaching Compound Only

Prepare absorbent article samples and determine the peroxygen bleaching compound concentration in each quadrant, using the methods described above. Calculate the distribution profile as follows:

$$Q 1 Profile = \frac{\begin{matrix}Measured Quantity Peroxygen \\ Bleaching Compound in Q 1\end{matrix}}{\begin{matrix}Measured Quantity Peroxygen \\ Bleaching Compound in Q 1 + Q 2 + Q 3 + Q 4\end{matrix}}$$

$$Q 2 Profile = \frac{\begin{matrix}Measured Quantity Peroxygen \\ Bleaching Compound in Q 2\end{matrix}}{\begin{matrix}Measured Quantity Peroxygen \\ Bleaching Compound in Q 1 + Q 2 + Q 3 + Q 4\end{matrix}}$$

$$Q 3 Profile = \frac{\begin{matrix}Measured Quantity Peroxygen \\ Bleaching Compound in Q 3\end{matrix}}{\begin{matrix}Measured Quantity Peroxygen \\ Bleaching compund in Q 1 + Q 2 + Q 3 + Q 4\end{matrix}}$$

$$Q 4 Profile = \frac{\begin{matrix}Measured Quantity Peroxygen \\ Bleaching Compound in Q 4\end{matrix}}{\begin{matrix}Measured Quantity Peroxygen \\ Bleaching Compound in Q 1 + Q 2 + Q 3 + Q 4\end{matrix}}$$

Profiles may be expressed as percentages by multiplying the calculated profile value by 100.

### Calculate the Distribution Profile—Odor Control System Comprising Peroxygen Bleaching Compound and Bleach Activator

Prepare absorbent article samples and determine the peroxygen bleaching compound concentration and bleach activator concentration in each quadrant, using the methods described above. Calculate the distribution profile as described above, substituting the combined weight of the peroxygen bleaching compound and the bleach activator for the measured quantity peroxygen bleaching compound in each quadrant.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a topsheet;
a backsheet;
an absorbent core between the topsheet and backsheet, the absorbent core comprising an odor control system;
wherein the absorbent core has a first quadrant, a second quadrant, a third quadrant, and a fourth quadrant; and
wherein the odor control system comprises a peroxygen bleaching compound, and the odor control system has a distribution profile greater than 5% in at least two of the four quadrants.

2. The absorbent article of claim 1, wherein the peroxygen bleaching compound has a distribution profile greater than 28% in at least two of the four quadrants.

3. The absorbent article of claim 1, wherein the odor control system comprises a coated peroxygen bleaching compound.

4. The absorbent article of claim 3, wherein the peroxygen bleaching compound is coated with one of the agents selected from the group consisting of a silicate, a borate, a sulfate, or combinations thereof.

5. The absorbent article of claim 1, wherein the odor control system has a distribution profile of approximately 25% in each of the first, second, third, and fourth quadrants.

6. The absorbent article of claim 1, wherein the odor control system comprises a bleach activator.

7. The absorbent article of claim 6, wherein the bleach activator is coated onto a core particle.

8. The absorbent article of claim 7, wherein the core particle is selected from the group consisting of sodium sulfate, sodium carbonate, and sodium phosphate.

9. The absorbent article of claim 1, wherein the peroxygen bleaching compound is selected from the group consisting of sodium percarbonate, sodium perborate, and combinations thereof.

10. The absorbent article of claim 3, wherein the peroxygen bleaching compound is selected from the group consisting of sodium percarbonate, sodium perborate, and combinations thereof.

11. The absorbent article of claim 6, wherein the bleach activator is selected from the group consisting of tetraacetyl ethylene diamine (TAED), benzoylcaprolactam (BzCL), 4-nitrobenzoylcaprolactam, 3-chlorobenzoylcaprolactam, benzoyloxybenzenesulphonate (BOBS), nonanoyloxybenzenesulphonate (NOBS), phenyl benzoate (PhBz), decanoyloxybenzenesulphonate (C₁₀-OBS), benzoylvalerolactam (BZVL), octanoyloxybenzenesulphonate (C₈-OBS), 4-[N-(nonaoyl) amino hexanoyloxy]-benzene sulfonate sodium salt (NACA-OBS), dodecanoyloxybenzenesulphonate (LOBS or C₁₂-OBS), 10-undecenoyloxybenzenesulfonate (UDOBS or C₁₁-OBS with unsaturation in the 10 position), and decanoyloxybenzoic acid (DOBA) perhydrolyzable esters, and mixtures thereof.

12. The absorbent article of claim 6, wherein the bleach activator has a leaving group selected from the group consisting of oxybenzenesulfonate (OBS), oxybenzoic acid (OBA), and valerolactam (VL).

13. The absorbent article of claim 6, wherein the bleach activator is selected from the group consisting of sodium nonanoyloxybenzenesulfonate (NOBS), tetraacetyl ethylene diamine (TAED), and combinations thereof.

14. The absorbent article of claim 6, wherein the bleach activator is present at a level of from about 0.005 g to about 0.2 g in the combined four quadrants.

15. The absorbent article of claim 6, wherein the bleach activator is present at a level of from about 0.001 g to about 0.05 g in the combined four quadrants.

16. The absorbent article of claim 6, wherein the bleach activator is present at a level of from about 0.005 g to about 0.010 g in at least one of the four quadrants.

17. The absorbent article of claim 1, further comprising a perfume raw material.

18. The absorbent article of claim 6, wherein the peroxygen bleaching compound and the bleach activator are present in a co-particle.

19. The absorbent article of claim 6, wherein the bleach activator is present as a particle, and wherein the bleach activator particles comprise a dye or pigment.

20. The absorbent article of claim 1, further comprising a non-activatable odor control system.

21. The absorbent article of claim 6, wherein the bleach activator is present as a particle, and wherein the bleach activator particles comprise a solid coating aid selected from the group consisting of acetates, sulfates, carbonates, borates, phosphates, and mixtures thereof.
